(19) United States
(12) Patent Application Publication
(10) Pub. No.: US 2025/0303414 A1
(43) Pub. Date: Oct. 2, 2025

(54) DEVICES, METHODS, AND SYSTEMS FOR MEASURING AND RECORDING A REACTANT ARRAY

(71) Applicant: SENSILL, INC., Concord, CA (US)

(72) Inventor: William Shea, Martinez, CA (US)

(73) Assignee: SENSILL, INC., Concord, CA (US)

(21) Appl. No.: 19/229,804

(22) Filed: Jun. 5, 2025

Related U.S. Application Data
(63) Continuation of application No. PCT/US2023/083076, filed on Dec. 8, 2023.

(51) Int. Cl. B01L 3/00; G01N 35/00; G01N 35/10

(52) U.S. Cl. CPC B01L 3/502715; G01N 35/00029; G01N 35/10; B01L 2200/16; B01L 2300/042

(57) ABSTRACT

Devices, systems, and methods include a system for analyzing reactant arrays. The system may include a cartridge including a reactant array and a device for analyzing the reactant array when the cartridge is received by or in the device. The cartridge may include a compartment for receiving a specimen that is in fluid communication with the reactant array and an opening configured to receive a sample portion of the specimen. The system may include the specimen having the sample portion configured to be positioned in the compartment and a non-sample portion extending out of the compartment.

### CROSS-REFERENCE TO RELATED APPLICATIONS

This application is a continuation of International Application No. PCT/US2023/083076, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for sensing and analyzing chemical substances, and methods for manufacturing and using such devices.

### BACKGROUND

A wide variety of devices have been developed for collection, storing, sensing, and analysis of samples. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example cartridge for use with a device for analyzing a colorimetric sensor array may include a colorimetric sensor array, a housing at least partially enclosing the colorimetric sensor array, wherein the housing may define a compartment for receiving a specimen, and an access opening extending from exterior the housing into the compartment for receiving the specimen.

Alternatively or additionally to any of the embodiments in this section, the housing may comprise a lid and the lid is adjustable between an open position to define the access opening and a closed position to block the access opening.

Alternatively or additionally to any of the embodiments in this section, the access opening may be a through hole extending from an exterior surface of the housing to the compartment for receiving the specimen.

Alternatively or additionally to any of the embodiments in this section, the through hole may be configured to receive a specimen comprised of a swab.

Alternatively or additionally to any of the embodiments in this section, the cartridge may further include a locking feature configured to prevent removal of the specimen from the housing.

In another example, a system for analyzing reactant arrays may include a cartridge, the cartridge includes a reactant array, a compartment for receiving a specimen that is in fluid communication with the reactant array, and an access opening configured to receive a sample portion of the specimen and a device for analyzing the reactant array when the cartridge is received in the device.

Alternatively or additionally to any of the embodiments in this section, the cartridge may include a housing at least partially enclosing the reactant array, defining the compartment for receiving the specimen, and the access opening through which the specimen is inserted into the compartment.

Alternatively or additionally to any of the embodiments in this section, the housing comprises a first component and a second component adjustable relative to one another between an opened position to define the access opening and a closed position to block the access opening.

Alternatively or additionally to any of the embodiments in this section, the housing is formed from a single housing component.

Alternatively or additionally to any of the embodiments in this section, the cartridge may include a lock having a locked position to prevent removal of the specimen from the cartridge.

Alternatively or additionally to any of the embodiments in this section, the lock may be automatically adjusted from an unlocked position to the locked position in response to insertion of the specimen in the cartridge.

Alternatively or additionally to any of the embodiments in this section, the device may be configured to automatically detect insertion of the cartridge in the device.

Alternatively or additionally to any of the embodiments in this section, the device may be configured to automatically detect insertion of the specimen in the cartridge.

Alternatively or additionally to any of the embodiments in this section, the device may be configured to collect light from the reactant array in response to the cartridge being positioned in the device.

Alternatively or additionally to any of the embodiments in this section, the system may further include the specimen, wherein the specimen may include the sample portion for positioning in the compartment and a non-sample portion extending out of the compartment.

Alternatively or additionally to any of the embodiments in this section, cartridge may be configured to sever a portion of the non-sample portion from the sample portion.

In another example, a cartridge for use with a device for analyzing reactant arrays may include a reactant array, a housing at least partially enclosing the reactant array, the housing defining a compartment for receiving a specimen, and an access opening extending from exterior of the housing into the compartment for receiving the specimen.

Alternatively or additionally to any of the embodiments in this section, the housing may include a first component and a second adjustable relative to one another between an open position to define the access opening and a closed position to block the access opening.

Alternatively or additionally to any of the embodiments in this section, the access opening may be a through-hole extending from an exterior surface of the housing to the compartment for receiving the specimen.

Alternatively or additionally to any of the embodiments in this section, the through-hole may be configured to receive the specimen and the specimen comprises a swab.

Alternatively or additionally to any of the embodiments in this section, the cartridge may further include a lock configured to prevent removal of the specimen from the housing.

Alternatively or additionally to any of the embodiments in this section, the lock may be configured to adjust from an unlocked position to a locked position in response to receiving the specimen in the compartment.

Alternatively or additionally to any of the embodiments in this section, the cartridge may further include a blade configured to sever the specimen received in the compartment.

Alternatively or additionally to any of the embodiments in this section, the cartridge may be configured to fluidly isolate the compartment and the reactant array from fluid exterior of the housing.

In another example, a method of analyzing reactant arrays may include inserting a cartridge into a device, where the cartridge includes a compartment and a reactant array and the device is configured to analyze the reactant array by collecting light through the cartridge, inserting a specimen into the cartridge, where the specimen includes a sample portion, the compartment is in fluid communication with the reactant array, and the sample portion is positioned in the compartment of the cartridge, and collecting, with the device, light from the reactant array after the cartridge is inserted into the device.

Alternatively or additionally to any of the embodiments in this section, the method may further include locking the specimen in the cartridge in response to receiving the specimen in the cartridge.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is a schematic diagram of an illustrative computing system;

FIG. 3 is a schematic diagram of an illustrative sensing system in a hand of a user;

FIG. 4 is a schematic perspective view of an illustrative cartridge;

FIG. 5 is a schematic perspective view of the illustrative cartridge of FIG. 4, with a specimen partially inserted therein;

FIG. 6 is a schematic perspective view of the illustrative cartridge of FIG. 4, with the illustrative cartridge in a closed position and with the specimen partially inserted therein;

FIG. 7 is a schematic perspective view of an illustrative cartridge, with the illustrative cartridge in an opened position;

FIG. 8 is a schematic perspective view of the illustrative cartridge of FIG. 7, with a specimen inserted therein;

FIG. 9 is a schematic perspective view of the illustrative cartridge of FIG. 7, with the cartridge in a closed position and with the specimen inserted therein;

FIG. 10 is a schematic perspective view of an illustrative cartridge, with the illustrative cartridge in an opened position and a specimen inserted therein;

FIG. 11 is a schematic perspective view of the illustrative cartridge of FIG. 10, with the illustrative cartridge in a closed position and with the specimen inserted therein;

FIG. 12 is a schematic perspective view of an illustrative cartridge, with the illustrative cartridge in an opened position and a specimen inserted therein;

FIG. 13 is a schematic cross-section view of the illustrative cartridge of FIG. 12, with a specimen inserted therein; and

FIG. 14 is a schematic diagram of an illustrative technique for analyzing a reactant array.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of volatile compounds (e.g., volatile organic compounds (VOCs)) and/or gasses, which may or may not be hazardous, may be sensed, analyzed, and/or monitored. Sensing, analyzing, and/or monitoring of fluids with analytes (e.g., non-volatile or volatile compounds, gases, liquids, and/or other fluids) may utilize absorption and/or reflectance measurements of reactants exposed to such fluids for any purpose including, but not limited to, diagnostic hazard warning, manufacturing processes or quality control, record keeping, archival purposes, product development, product-consumer matching, etc.

In some cases, VOCs and/or gasses may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gasses exhaled or emitted, excreted, emanated, released, and/or secreted from a subject (e.g., humans, animals other than humans, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, ear canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring fluids (e.g., for analytes of interest) may be configured to accurately detect and record a reactant array (e.g., a reactant array of a colorimetric sensor array (CSA) or other suitable reactant array) spectral response to exposure to the fluids. The systems may utilize techniques for non-invasively detecting one or more analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a CSA to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

An analysis system (e.g., a CSA analysis system) may include an enclosure or cartridge containing a reactant array (e.g., a CSA having a reactant array) and a device for reading or otherwise analyzing the CSA in the cartridge before, during, and/or after the reactant array is exposed to a fluid. The device for analyzing the reactant array (e.g., a reader device) may be or may include a spectrometer or other suitable image or light collector/sensor. In some instances, the device may be configured to be handheld. Further, the device for analyzing the reactant array may be configured to be simple to use, minimize human error, reduce cross contamination of samples, and reduce human risk of exposure to analytes received at the device.

In operation, the enclosure cartridge containing a reactant array may be placed into the device for analyzing the reactant array (e.g., via an opening in the device) during and/or through pre and post exposure to fluid for optimal reactant array image/reflectometer analysis. That is, analysis of the reactant array in the cartridge may include analyzing the reactant array prior to exposure to the fluid, during exposure to the fluid, and/or after exposure to the fluid is complete. Once the analysis of the reactant array in the cartridge is complete, the cartridge may be removed from the device for analyzing the reactant array and discarded. Having the cartridge be disposable may prevent cross-contaminations of the reactant array and erroneous analyses (e.g., due to the previous fluids persisting in the tubing and reflowing over the reactant array, undesirably flowing onto a human, etc.).

The cartridge may be any suitable component including one or more reactant arrays (e.g., where the reactant arrays may be permanent or removable from the cartridge) and that may be configured to expose the one or more reactant arrays to fluids from a target area or site. In one example, the cartridge may be a flow cell and may have an input port for receiving fluid (e.g., a fluid from a target area or site) to pass over and/or through the one or more reactant arrays and an output port for outputting the fluid passed through and/or over the one or more reactant arrays. In the example, the fluid received may be pressurized via pressurized fluid (e.g., N₂, etc.) from an external source and/or pressurized via a pump in communication with a fluid path between the input port and the output port of the cartridge.

In an additional or alternative configuration, the cartridge may be configured to receive a specimen including a sample from a target area or site. In some cases, the cartridge may include a compartment configured to receive at least a portion of the specimen with the sample thereon and/or therein.

When the cartridge is configured to receive a specimen, the specimen received may be or may include any suitable specimen substrate configured to collect or carry samples or fluids from a subject. The specimen may be or may include a specimen substrate with a sample from a target site. Example suitable specimen substrates may include, but are not limited to, swabs, swabs on a stick (e.g., a Q-tip), sponges, sample plates, test strips, needle sticks, syringes, etc.

The compartment of the cartridge configured to receive at least the specimen substrate may be accessed in any suitable manner. For example, the compartment may be accessed via an access opening extending from exterior of the housing into the compartment for receiving a specimen. Example access openings include, but are not limited to, one or more openings extending through the housing defining or at least partially defining the compartment (e.g., a through hole extending from an exterior surface of the housing to the compartment), a lid (e.g., a clam shell configuration or other suitable lid configuration) configured to adjust (e.g., pivot, etc.) between an open position of the cartridge that provides access to the compartment and a closed position of the cartridge that seals the compartment and/or blocks access to the compartment through the access opening, and/or other suitable configurations of the cartridge configured to receive and secure the specimen in the cartridge. When the specimen includes a swab on a stick as a specimen substrate, for example, the cartridge may be configured to receive the swab on a stick or other suitable specimen in the compartment via a through hole such that the stick extends out of a through hole opening of the cartridge.

Once a specimen is received in the cartridge, the cartridge may be configured to maintain the specimen in the compartment or allow the specimen to be removed from the cartridge prior to, during, and/or after analysis of the sample of the specimen. In some cases, the compartment of the cartridge may be sealed in response to receiving the specimen, but this is not required. After a specimen is received in the cartridge, the sample or fluids thereon or therein may emanate from the specimen and/or permeate through the one or more CSAs within the cartridge.

In some cases, the cartridge may include a locking feature configured to lock the specimen in the cartridge. In one example of a locking feature, the cartridge may include a locking feature that is configured to cut the stick of the specimen at an outer surface of the opening or through hole and then seal the opening. Alternatively or additionally, a locking feature configured to cut the stick or other portion of the specimen may be a portion of a lid that is configured to sever the specimen inserted in the cartridge into a sample portion that is secured in the compartment of the cartridge and a non-sample portion that is removed from the cartridge. Other suitable locking features include, but are not limited to, features that engage a received specimen, prevent removal of a received specimen after placement of the specimen in the compartment, and/or other suitable locking features configured to prevent removal of the specimen from the housing and/or encourage single-use of the cartridge (e.g., to prevent cross-contamination of samples).

When the cartridge includes a lid, the locking feature may be configured to lock the lid in a closed position after a specimen has been received in the cartridge. In some cases, when the lid is configured to be locked in the closed position after a specimen has been received in the cartridge, the locking feature may permanently lock the lid in the closed position, where the permanent lock may be triggered by receiving the specimen in the cartridge and effected by closing the lid, but this is not required and other suitable permanent lock systems may be utilized.

A cartridge configured to receive a specimen including a sample from a target site relative to cartridges configured to receive samples or fluids in other suitable manners, as discussed herein for example (e.g., through a fluid path extending between an input port and an output port of the cartridge), may result in mitigating cross-contamination, reducing setup complexity by not requiring a tube configuration, reducing site preparation time, minimizing analysis time for fluids and/or samples, increasing concentration of samples and/or fluids collected from a target area or site (e.g., via use of a specimen), among other benefits. Moreover, when a swab or other common sample collection devices are used as a specimen substrate for collecting samples from a target area or site, a clinician may be using a collection device that it is familiar with, which may allow for effective collection and maintenance of samples on the specimen due to using known techniques common to a clinical setting.

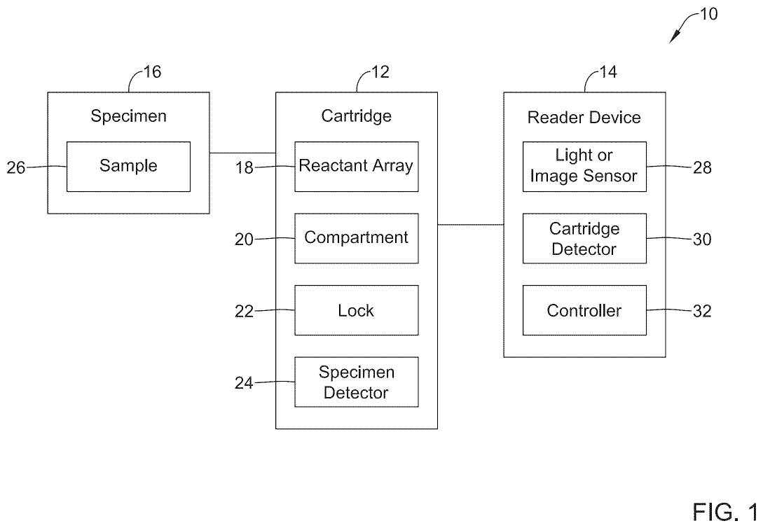

Turning to the Figures, FIG. 1 depicts a schematic diagram of an illustrative system 10 for analyzing a reactant array. Among other components the system 10 may include a cartridge 12 having the reactant array 18 and a reader device 14 configured to monitor and/or analyze the reactant array 18. In some configurations, the system 10 may include a specimen 16 configured to collect a sample from a target area (e.g., a wound, pollen from a flower, an infection, an exhalation from a subject, a sweat gland, etc.) for analysis using the cartridge 12 and/or the reader device 14.

The cartridge 12 may have any suitable configuration and may include any suitable components configured to facilitate receiving the specimen 16, sensing analyte from the specimen 16, and interfacing with the reader device 14. Example components of the cartridge 12 include, but are not limited to, one or more reactant arrays 18, one or more compartments 20, one or more locks 22, one or more specimen detectors 24, and/or one or more other suitable components and/or configurations. Components that the cartridge 12 may include but are not depicted in FIG. 1 include, but are not limited to, a housing, a window for viewing the reactant array 18, a blade for engaging the specimen 16, one or more gaskets or other features for hermetically sealing the one or more compartments, one or more access openings extending between the compartments and exterior of the housing, one or more valves configured to seal the access opening, one or more doors or lids, one or more pumps for pumping fluid from a location of the specimen 16 to the reactant array 18, one or more fluid paths or passages, tubing, one or more diaphragms or membranes, one or more single-use components, and/or other suitable components.

Although not depicted in FIG. 1, the cartridge 12 may include the housing, where the housing may be configured to at least partially enclose or house all or one or more of the reactant arrays 18, the compartments 20, the locks 22, and the specimen detectors 24. The housing may be a single component with an opening between the one or more compartments 20 and an exterior of the cartridge 12 and/or a plurality of components defining the opening between the one or more compartments 20 and the exterior of the cartridge 12.

The housing may be formed in any suitable manner. In some examples, the housing may be formed with one or more molding techniques, injection molding techniques, welding techniques, ultrasonic welding techniques, three-dimensional (3D) printing techniques, and/or other suitable techniques.

The housing of the cartridge 12 may be formed from any suitable material. Example suitable materials include, but is not limited to, polymers, metals, glass, and/or other suitable types of materials.

The housing of the cartridge 12 may have any suitable shape and/or size configured to receive a specimen and facilitate analyzing the reactant array in the cartridge 12 using the reader device 14. For example, the cartridge 12 may have a cube shape, an elongated shape, a rectangular shape, an oval shape, a rounded shape, a circular shape, a ball shape, and/or other suitable shape.

The reactant array 18 may be any suitable array of one or more reactants (e.g., analyte sensitive material) and the reactants of the reactant array 18 may be formed from any suitable material. The cartridge 12 (e.g., the housing of the cartridge 12) may be configured to entirely or at least partially house or enclose the reactant array 18 (e.g., the CSA including the reactant array 18). In some cases, the reactant array 18 may be part of or form a colorimetric sensor array (CSA), but other suitable configurations are contemplated. Additionally or alternatively, the reactant array 18 be formed may applying the material of the reactants to an entirety or at least a portion of the internal surface of the cartridge 12 (e.g., surfaces of the compartments 20) and the applied reactant material may be a continuous pattern, partitioned, segmented, perforated, and/or one or more other suitable designs or configurations.

The material of the reactants of the reactant array 18 may be reversible (e.g., reusable), semi-reversible, or non-reversible (e.g., single use). In some examples, the material of the reactants may be an optically responsive chemical material (e.g., a chemoresponsive material) that changes color in response to detecting one or more analytes (e.g., volatile compounds gasses, liquids, and/or other fluids) in a fluid to which the reactants are exposed, but other suitable material is contemplated. Example suitable materials for reactants include dyes from, but not limited to, the following classes: Lewis acid/base dyes (e.g., metal containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example material for the reactants may be a silver nanoparticle material. Other suitable materials for the reactants are contemplated, including reactant material other than a printed dye or an optically responsive chemical material.

The reactants of the reactant array 18 may be applied to a substrate. When the reactant array 18 forms or is otherwise part of a CSA, the substrate may or may not be part of the CSA. Additionally or alternatively, the substrate may be the cartridge 12 (e.g., the housing of the cartridge and/or other suitable component of the cartridge) or a component configured to be received in the cartridge 12 (e.g., in one or more of the compartments 20).

The reactants of the reactant array 18 may be applied to a substrate in any suitable manner. In one example, the reactants may be applied to the substrate by printing the reactants (e.g., the material of the reactants) on the substrate. When printed, any suitable printing techniques may be utilized including, but not limited to, pin transfer, inkjet, silkscreen, and/or other suitable application techniques.

The reactants may be applied to the substrate randomly and/or to form one or more patterns. Example configurations of the reactants of the reactant array applied to the substrate include, but are not limited to, grid patterns of rows and columns, concentric rings, color matching of a color of printed dye material with a color of a substrate material prior to interactions of the reactants with analyte, patterns that result in identifiable shapes when the analyte sensitive material reacts to a particular analyte, other suitable configurations, and/or combinations thereof.

The one or more compartments 20 of the cartridge 12 may be entirely or at least partially defined by the housing of the cartridge 12. The one or more compartments 20 may be in fluid communication with one or more access openings extending from an exterior side of the cartridge 12 (e.g., the housing of the cartridge 12) into the one or more compartments 20. In some examples, the access opening may be configured to receive the specimen 16 for positioning in the one or more compartments 20.

The one or more compartments 20 may include any suitable configuration of compartments 20. For example, the compartments 20 may define a fluid path from a location of the specimen 16 to the reactant array 18, may be a single compartment 20 configured to include the reactant array 18 and receive the specimen 16, may be or include a plurality of compartments fluidly coupled to allow fluid to pass between a location of the specimen 16 and a location of the reactant array 18, and/or the compartment(s) 20 may have one or more other suitable configurations. Although not depicted, the one or more compartments may be or may include one or more tubes defining at least part of a fluid path from a location of the specimen 16 to reactant array 18.

Once the specimen 16 is received in or at the cartridge 12, the cartridge 12 may maintain the specimen 16 in or at the compartment 20 or may allow the specimen 16 to be removed from the cartridge 12 prior to, during, and/or after analysis of a sample (e.g., fluids from the sample) on or of the specimen 16. In some configurations, the compartment 20 of the cartridge 12 may be sealed in response to receiving the specimen 16, but other configurations are contemplated. After a specimen is received in the cartridge, fluids thereon or therein may emanate from the specimen 16 and/or permeate through the reactant array 18 within the cartridge 12.

The lock 22 may have any suitable configuration. In some examples, the lock 22 may be any suitable any suitable component that is configured to selectively block access to the compartments 20 via the access opening. When the housing of the cartridge 12 includes multiple components adjustable relative to one another between an opened position to define the access opening and a closed position to block access to the compartments 20 via the access opening, the lock may be configured to fix the multiple components relative to one another (e.g., in the closed position and/or other suitable position).

The lock 22 may be configured to secure the specimen 16 relative to the cartridge 12. In one example, the cartridge 12 may include a blade or blade surface that is configured to cut the specimen into two sub-components and then seal the access opening. Alternatively or additionally, the lock 22 including the blade or blade surface configured to cut the specimen may be a portion of a component of the housing of the cartridge 12 that is configured to sever the specimen inserted in the cartridge 12 into a sample portion that is secured in the compartment of the cartridge 12 and a non-sample portion that is removed from the cartridge. In some examples, receiving the specimen 16 in the access opening may trigger the lock 22 to close off the access opening and/or engage the specimen to prevent removal of the specimen 16. Other suitable features of the lock 22 include, but are not limited to, features that engage a received specimen 16, features that prevent removal of a received specimen 16 after placement of the specimen 16 in the compartment 20, spring loaded doors, automated doors, bolts, latches, magnets, and/or other suitable features of locks configured to prevent removal of the specimen 16 from the housing, encourage single-use of the cartridge (e.g., to prevent cross-contamination of samples), and/or coupling multiple components of a housing to one another and/or the specimen 16.

In some examples, the lock 22 may be irreversible such that the specimen 16 cannot be removed from the cartridge 12 and the lock 22 facilitates ensuring the specimen 16 and the cartridge 12 are only used to test one sample. In other examples, the lock 22 may be reversible to allow the specimen 16 to be rearranged relative the cartridge 12.

Any suitable type of locks 22 may be utilized. Example suitable types of locks 22 include, but are not limited to, automatically actuated locks, manually actuated locks, spring loaded locks, latches, dead bolts, friction fit locks, and/or other suitable types of locks that are configured to block the access opening and/or engage the specimen to prevent removal and/or adjustment of the specimen 16 relative to the cartridge 12.

The cartridge 12 may include one or more specimen detectors 24. In some examples, the specimen detector 24 may be configured to detect when a specimen is proximate and/or received in the cartridge 12 and may or may not be part of the lock 22. In one example, the specimen detector 24 may be a pin or button or other component in communication with a spring-loaded lock 22, where the pin or button or other component detects the specimen and releases a spring of the lock 22 to lock the specimen 16 within the cartridge 12. Alternatively or additionally, the specimen detector 24 may be an electronic sensor or detector and/or other suitable type of detector that may provide an indication to the reader device 14 indicating that a specimen has or has not been received in the cartridge 12 (e.g., has or has not been properly received in the cartridge 12).

In addition to or as an alternative to the lock 22 and/or the specimen detector 24, the cartridge 12 may include a single-use component configured to prevent contamination due to reuse of a cartridge. In some cases, the single-use component may be a mechanical, electrical, electromechanical, optical, chemical, magnetic, and/or other suitable type of single-use feature that changes in response to being used with a single-use or reusable reader device 14 for analyzing the reactant array 18. In one example of a single-use component for a cartridge 12, an electronic single-use component may be imbedded within the cartridge 12 and either recognized by the reader device 14 as being intact or broken to indicate whether the cartridge 12 is new or has been used, respectively. Example electronic single-use components include, but are not limited to a memory devices, radio-frequency identification (RFID) devices, a fuses, and/or other suitable electronic single-use components. When a memory device is used as a single-use component of the cartridge 12, the memory device may be accessed by the reader device 14 and modified by the reader device 14 after the cartridge 12 has been inserted into the reader device 14. When a fuse is used as a single-use component, the fuse may be broken (e.g., via excessive current or in other suitable manners) after the reader device 14 analyzes the reactant array 18, where the reader device 14 may be configured to identify the broken fuse and the cartridge 12 with the broken fuse may be prevented from being used in a subsequent analysis. Further, the single-use component may be or may include at least one visual indicator that may change state after use in the reader device 14 and that may be captured by an image capture sequence of the reader device 14, where the reader device 14 prevents use of the cartridge 12 if it determines from the indicator that the cartridge 12 has been used.

In some cases, the cartridge 12 may be reusable. For example, in instances when the reactant array 18 and/or other components of the cartridge 12 that may be spent or contaminated with a received fluid, the reactant array 18 and/or other spent or contaminated components of the cartridge 12 may be removed from the cartridge 12 and the remaining components of the cartridge 12 may be reused after cleaning, as needed. In some cases, the reactant array 18 include reversible reactant material and the reactant array 18 may be reused with other components of the cartridge 12.

The specimen 16 may be configured to collect a sample from a target area, where the sample is configured to exude fluids. In some examples, the specimen 16 may include a sample portion 26 configured to contact a target area to collect a sample that exudes fluid. The specimen 16 may include a non-sample portion along with the sample portion 26, where the non-sample portion may be handled by a user for positioning the specimen at the cartridge and/or to collect the sample. When the specimen 16 is positioned within a compartment 20 of the cartridge 12, the sample portion 26 of the specimen 16 may be inserted into the compartment 20 and the non-sample portion may extend out of the compartment 20, but other suitable configurations are contemplated.

The specimen 16 may be any suitable type of specimen component configured to collect a sample from a target area. Example suitable types of specimens 16 include, but are not limited to, swabs, swabs on a stick (e.g., a Q-tip), containers, sponges, sample plates, test strips, containers, needle sticks, syringes, and/or other suitable types of specimens configured to support a sample.

The reader device 14 may include one more suitable components for reading and/or analyzing the reactant array 18. Example suitable components of the reader device 14 include, but are not limited to, illumination components, light collection components, one or more light or image sensor 28, one or more cartridge detector 30, one or more controllers 32, one or more light sources, one or more sets of lenses, one or more motors, one or more pumps, one or more buttons, one or more user interfaces, one or more displays, and/or other suitable components. In some examples, the reader device 14 may be a bench top device or a handheld device. In some cases, the reader device 14 may be isolated from the sample of the specimen 16 and may be configured for reuse.

The reader device 14 may be powered with any suitable power source. Example suitable power sources for powering the reader device 14 include, but are not limited to, battery power in the reader device 14, solar power at the reader device 14, wall or line power, and/or other suitable power sources. In some examples, to facilitate forming the reader device 14 with a handheld configuration, the reader device 14 may be powered by one or more batteries and/or by solar power.

The light or image sensor 28 may be and/or may include one or more light collectors of any suitable type. Example suitable types of light collectors may include, but are not limited to, a light sensor, an image sensor, an n-dimensional sensory array (e.g., where “n” equals 1, 2, etc.), a linear 2D light detector array image sensor, light detector array image sensor, a spectrometer, a refractometer, a charge-coupled device (CCD) image sensor, complementary metal-oxide semiconductor (CMOS) image sensor, contact image sensor (CIS), color contact image sensor (CCIS), a camera, other suitable light collectors, and/or combinations of light collectors. In one example, the light collector may include or may be a spectrometer configured to measure photons collected from (e.g., reflected, transmitted, and/or otherwise received from) the reactant array. Utilizing a spectrometer may facilitate sensing wavelengths of light with high resolution in the nanometer range and may provide a continuous set of data over the wavelength range, which allows for a sensitive analysis of the data to identify components of a fluid to which the reactant array 18 was exposed relative to when other light collectors are used. In another example, the light collector may include a 2D pixel array image sensor configured to record multiple spatial interferograms in a pixel array direction of an interferogram representing a Fourier transform of the reactant array 18, which may provide sufficient sensitivity, while being compact and cost-effective.

The reader device 14 may include one or more cartridge detectors 30. In some examples, the cartridge detector 30 may be configured to detect when the cartridge 12 is proximate and/or received in the reader device 14. In one example, the cartridge detector 30 may be a pin or button or other component, where the pin or button or other component physically or mechanically engages the cartridge 12 as the cartridge 12 is inserted into or positioned at the reader device 14 and in response, the pin or button or other component adjusts and provides a mechanical and/or electrical (e.g., completes a circuit, etc.) indication that the cartridge 12 has been received. Alternatively or additionally, the cartridge detector 30 may be an electronic sensor or detector and/or other suitable type of detector that may provide an indication to the reader device 14 indicating that the cartridge 12 is proximate and/or has or has not been received in the reader device 14 (e.g., has or has not been properly received in the reader device 14). In some cases, the electronic sensor or detector may sense a signal from the cartridge 12 (e.g., an RF signal), sense the cartridge 12 breaking a circuit of the reader device 14, completing a circuit of the reader device 14 and/or the cartridge 12, and/or sense a presence of the cartridge 12 in one or more other suitable manners.

In addition to or as an alternative to the cartridge detector 30, the reader device 14 may include a single-use component. The single-use component of the reader device 14 may include a feature configured to electrically, mechanically, or electrically and mechanically modify the cartridge 12 such that the cartridge 12 will not be used more than once by the reader device 14 (e.g., in more than one test by the reader device 14). Example single use components include, but are not limited to, a component configured to write to an RFID tag of the cartridge 12, a component configured to mechanically alter the cartridge 12 to prevent recoupling of the cartridge 12 after the cartridge 12 has been removed from the reader device 14, a camera configured to read a code (e.g., a bar code, QR code, alphanumerical code, color code, etc.) on a surface of the cartridge 12 or an RFID reader configured to read a code from an RFID of the cartridge and add the code or other identifying material to a list of used cartridges in memory of the reader device 14 and/or in communication with the reader device 14.

The cartridge detector 30 and/or other suitable detector of the reader device 14 may be configured to detect when the specimen 16 is within or at the cartridge 12. For example, the cartridge 12 may be inserted into the reader device 14 before the specimen 16 is within the cartridge 12 and the cartridge detector 30 may detect when the specimen 16 is inserted into the cartridge 12 after the cartridge 12 is inserted into the reader device 14. Upon sensing the cartridge 12 in the reader device 14 and the specimen 16 in the cartridge 12, the reader device 14 may initiate a light or image collection from the reactant array 18 as part of a fluid analysis test of a sample on the specimen 16 and/or a request for input from a user to perform one or more steps of the fluid analysis test.

The controller 32 of the reader device 14 may be configured to control operations of the reader device 14 in response to receiving one or more control signals and/or use inputs. The controller 32 may store captured data at the reader device 14 and/or send data to a remote storage component for storage and the controller 32 may use stored captured data to analyze the reactant array 18. Further the controller 32, may be implemented entirely on the reader device 14, partially on the reader device 14 and partially remotely, and/or entirely remotely (e.g., on a server or other suitable computing device, on the cartridge 12, on a user's mobile device, etc.)

The controller 32 of the reader device 14 may be coupled to one or more other electronic components of the system 10. For example, the controller 32 may be communicatively coupled with one or more of the illumination components, when included, the light or image sensor 28, the cartridge detector 30, the single-use component, and/or one or more other suitable components of the system 10 and/or remote components (e.g., servers, mobile devices, etc.) that may or may not be part of the system 10. In some examples, the controller 32 may be configured to receive an indication to initiate a fluid test (e.g., from a user via a user interface of or in communication with the controller 32, from the cartridge detector 30, etc.) and send coordinated control signals to one or more electronic components of the system 10.

The controller 32 may be configured to identify or may facilitate identifying a component of fluid in contact with the reactant array 18 and/or a condition of a target area based on measured (e.g., sensed and/or calculated) levels of light (e.g., interferograms, images, reflectance, etc.) or changes in light sensed or collected from the reactant array 18 with the light or image sensor 28. In some examples, the controller 32 may be configured to identify a component of fluid in contact with the reactant array 18 and/or a condition of the target area based on one or more of a timing of levels of light from the reactant array 18 and an absolute change between a level of light from the reactant array 18 at a time of or prior to inserting the sample portion 26 of the specimen 16 into or exposing the sample portion 26 to the compartment 20 of the cartridge 12 and at a predetermined time after initially inserting the sample portion 26 in the compartment 20, and levels of light from the reactant array 18 relative to predetermined or expected levels of light from the reactant array 18. The controller 32 may be configured to identify the component of the fluid in contact with the reactant array 18 and thus, a component of the sample at the sample portion 26 of the specimen 16 and/or a condition at a location from which the sample was taken (e.g., a wound, pollen from a flower, an infection, an exhalation from a subject, a sweat gland, etc.) based on light from the reactant array 18 that is received at the light or image sensor 28 in one or more additional or alternative manners.

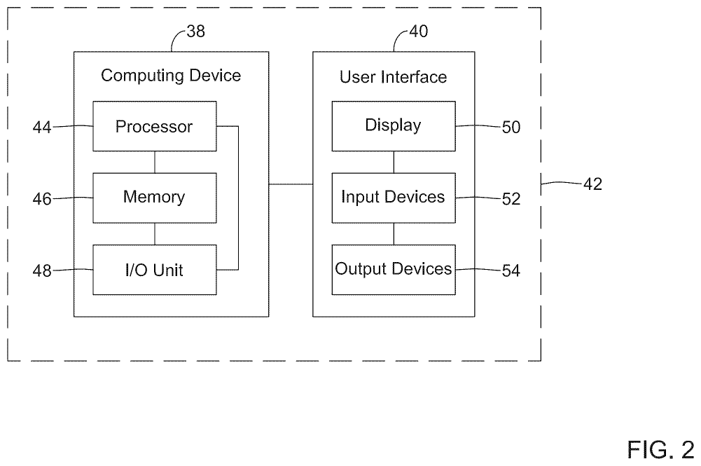

The controller 32 and/or other components of the system 10 may be or may include one or more computing devices including or coupled with one or more user interfaces. FIG. 2 depicts a schematic diagram of an illustrative computing device 38 and a user interface 40, where the computing device 38 and/or the user interface 40 may be entirely or partially housed in one or more housings 42 (e.g., a housing which may or may not house other components of the system 10). The housing 42 may be an optional component, as represented by the broken lines defining the housing 42 depicted in FIG. 2. Although various components are depicted as being included in the computing device 38 and the user interface 40, one more of the depicted components may be omitted and/or one or more additional or alternative components may be utilized.

The computing device 38 may be any suitable computing device configured to process data of or for the system 10 and may be configured to facilitate operation of the system 10. The computing device 38, in some cases, may be configured to control operation of the system 10 by establishing and/or outputting control signals to the light or image sensor 28 and/or other electronic components of the system 10 to run a test on fluid from the sample portion 26 of the specimen 16 that interacts with the reactant array 18 and/or monitor results of a test. In some examples, the computing device 38 may be part of the controller 32 and may communicate with other components over a wired or wireless connection, but other suitable configurations are contemplated. When the computing device 38, or at least a part of the computing device 38, is a component separate from a structure of the controller 32, the computing device 38 may communicate with electronic components of the system 10 over one or more wired or wireless connections or networks (e.g., LANs and/or WANs). In some cases, the computing device 38 may communicate with a remote server or other suitable computing device.

The illustrative computing device 38 may include, among other suitable components, one or more processors 44, memory 46, and/or one or more input/output (I/O) units 48. Example other suitable components of the computing device 38 that are not specifically depicted in FIG. 2 may include, but are not limited to, communication components, a touch screen, selectable buttons, and/or other suitable components of a computing device. As discussed, one or more components of the computing device 38 may be separate from the controller 32 and/or incorporated into the components of the controller 32.

The processor 44 of the computing device 38 may include a single processor or more than one processor working individually or with one another. The processor 44 may be configured to receive and execute instructions, including instructions that may be loaded into the memory 46 and/or other suitable memory. Example components of the processor 44 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 46 of the computing device 38 may include a single memory component or more than one memory component each working individually or with one another. Example types of memory 46 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 46 may be or may include a transitory or a non-transitory computer readable medium. The memory 46 may include instructions stored in a transitory state and/or a non-transitory state on a computer readable medium that may be executable by the processor 44 to cause the processor 44 to perform one or more of the methods and/or techniques described herein. Further, in some cases, the memory 46 and/or other suitable memory may store data received from the light or image sensor 28 and/or other components of or in communication with the system 10.

The I/O units 48 of the computing device 38 may include a single I/O component or more than one I/O component each working individually or with one another. Example I/O units 48 may be or may include any suitable types of communication hardware and/or software including, but not limited to, communication components or ports configured to communicate with electronic components of the system 10 and/or with other suitable computing devices or systems. Example types of I/O units 48 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 48.

The user interface 40 may be configured to communicate with the computing device 38 via one or more wired or wireless connections. The user interface 40 may include, among other components, one or more display devices 50, one or more input devices 52, one or more output devices 54, and/or one or more other suitable features. Although not depicted, the user interface 40 may include one or more indicators (e.g., light emitting diodes (LEDs), LED linear arrays, numbers, etc.) In some examples, the user interface 40 may be part of or may include the computing device 38. Alternatively or additionally, the user interface 40 may be part of a mobile device or remote computing system.

The display 50 may be any suitable display. Example suitable displays include, but are not limited to, touch screen displays, non-touch screen displays, liquid crystal display (LCD) screens, LED displays, head mounted displays, virtual reality displays, augmented reality displays, a mobile device display, and/or other suitable display types.

The input device(s) 52 may be and/or may include any suitable components and/or features for receiving user input via the user interface 40. Example input device(s) 52 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 52 may include a touch screen that allows for setting set points, initiating a fluid or target area analysis test, adjusting between screens (e.g., a testing screen, a data analysis screen, a results screen, etc.), and/or allows for taking one or more other suitable actions.

The output device(s) 54 may be and/or may include any suitable components and/or features for providing information and/or data to users and/or other computing components. Example output device(s) 54 include, but are not limited to, displays, speakers, vibration systems, tactile feedback systems, optical outputs, and/or other suitable output devices.

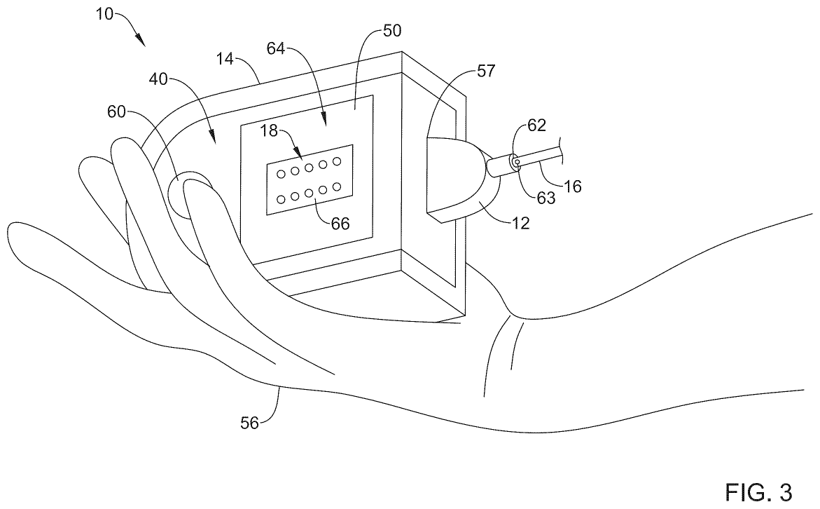

FIG. 3 schematically depicts an illustrative configuration of the system 10 configured to be held in a hand 56 of a user. Although the system 10 may take on a variety of different handheld configurations (and/or non-handheld configurations), the system 10 depicted in FIG. 3 may include the cartridge 12 inserted into the reader device 14 through an access opening 57. The cartridge 12 includes a specimen 16 inserted through an access opening 62.

In some configurations, the specimen 16 may include a stop 63 that may be configured to abut a portion of the cartridge 12 defining the access opening 62 and limit insertion movement of the specimen 16. The stop 63 may be configured along the specimen 16 to position the sample portion 26 of the specimen 16 at a desired location in the cartridge 12 (e.g., prevent the sample portion 26 from engaging an interior wall of the cartridge 12). Further, the specimen 16 may include a perforation or other suitable configuration proximate the stop 63 that may facilitate breaking of the specimen at or proximate the stop. Other suitable configurations are contemplated.

The user interface 40 of the reader device 14 depicted in FIG. 3 may include the display 50 and one or more buttons 60. The user interface 40 may include additional and/or alternative components or features including, but not limited to, one or more indicators (e.g., LEDs, LED linear arrays, numbers, etc.) configured to indicate (e.g., as an alert, etc.) a result of a test, a test has been initiated, a test has been completed, a user is to perform an action, an action has been completed, and/or other suitable indications. Additionally or alternatively, the user interface 40 may be on or part of a remote computing device, such as a mobile device, control station, web page, mobile application, and/or other suitable remote computing device. In some examples, the user interface 40 may be entirely omitted from the reader device 14 or one or more components of the user interface 40 discussed herein may be omitted from the reader device 14.

The display 50 (e.g., a touchscreen display or a non-touchscreen display) may depict an image 64 captured by the reader device 14 (e.g., captured by the light or image sensor 28). The image 64 may be a live image and/or a photograph or image captured at a previous time. As schematically depicted in FIG. 3, the image 64 may be a live image of the CSA 66 including the reactant array 18. Further, the display 50 may display material other than the image 64 including, but not limited to, instructions for testing a fluid, a test status (e.g., a progression of steps in an analysis of the reactant array 18), a system status, results of an analysis of the reactant array 18, marketing indicia, brand indicia, videos, user pictures, art work, etc.

The one or more buttons 60 may be selected by a user to cause the reader device 14 and/or the cartridge 12 to take one or more actions. For example, a user may interact with the one or more buttons 60 to initiate a pump of the reader device 14 and/or of the cartridge 12, power on and/or off the reader device 14, initiate a motor of the reader device 14, initiate an analysis of the reactant array 18, initiate the light or image sensor 28 to take an image or capture light, eject the cartridge 12 and/or the specimen 16, mark the cartridge 12 used, initiate the display 50, and/or to cause the reader device 14 and/or the cartridge 12 to take one or more other suitable actions.

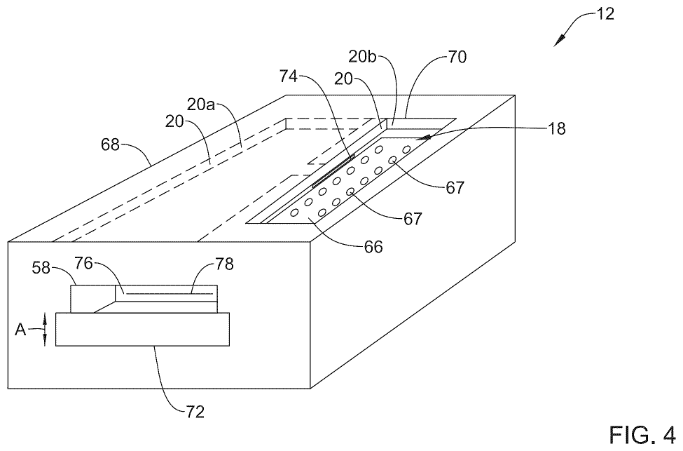

FIGS. 4-6 schematically depict an illustrative configuration of the cartridge 12 including the access opening 58 and a housing 68 having a transparent portion 70 (e.g., a window formed from glass, polymer, and/or other suitable transparent material) through which the reader device 14 may view, collect light, and/or image the reactant array 18 positioned within a compartment 20. In some examples, the housing 68 may be formed from a single housing component or structure with the access opening 58 being the only access point to the compartment 20 in which the reactant array 18 and/or the specimen 16 may be located, but other suitable configurations are contemplated. Although not required, the cartridge 12 may include a door 72 configured to adjust in the directions of arrows A or in other suitable directions between an opened position at which the access opening 58 may be accessible and a closed position at which the access opening 58 is not accessible or is blocked. In some configurations, the door 72 may be configured to fluidly seal (e.g., hermetically seal) the access opening 58 and/or the compartments 20 when in the closed position.

In addition to or as an alternative to utilizing the door to fluidly seal (e.g., hermetically seal) the compartment(s) 20 from exterior of the housing 68, one or more valves 76 may be positioned at or in the access opening 58. In some examples, as depicted in FIG. 4, a single valve 76 (e.g., a check valve and/or other suitable type of valve) may be located in the access opening 58, where the single valve 76 may include a slit 78 or other opening biased closed and that may be opened in response to engagement with the specimen 16. When the specimen 16 extends through the valve 76, the valve 76 may be configured to seal around the specimen 16. When the specimen 16 is to be positioned entirely within the compartment(s) 20, the valve 76 may be configured to re-seal the access opening 58 after the specimen 16 has been inserted therethrough. To prevent the valve 76 from engaging a sample portion of the specimen 16, the specimen 16 may include a distal blocker configured to engage and open the valve 76 to prevent the valve 76 from engaging a sample portion of the specimen 16 and/or the valve 76 may be configured to open or be opened without the specimen 16 engaging the valve 76.

Alternatively or additionally to utilizing one or more valves 76, one or more seals (e.g., a single-use seal, such as a foil seal, a burstable membrane, and/or other suitable seals) may be utilized at or proximate to the access opening 58 to seal the compartments 20 from the ambient environment and mitigate contamination of the reactant array 18. In some examples, a single-use seal may extend across the access opening 58 and may be pierced or broken to remove the seal from the access opening 58. In some examples, the single-use seal may be a peel-away seal configured to be peeled off of the access opening 58 prior to inserting the specimen 16 through the access opening 58.

As depicted in FIG. 4, the housing 68 may be formed from a single housing component. The door 72, however, may be considered part of the housing 68, but this is not required. In some examples, the door 72 may be a first component of the housing 68 and the portion of the housing 68 defining the compartment(s) 20 may be a second component of the housing 68.

The housing 68 may include or define one or more compartments 20. As depicted in FIGS. 4-6, the housing 68 may have a first compartment 20a for receiving the sample portion 26 of the specimen 16 and a second compartment 20b for containing the reactant array 18 (e.g., which may be part of the CSA 66). When two or more compartments 20 are utilized in the housing 68, the compartments 20 may be fluidly connected by one or more fluid passages 74 such that fluid may pass from one compartment 20 (e.g., the first compartment 20a) to another (e.g., the second compartment 20b).

FIG. 4 schematically depicts a perspective view of the cartridge 12 with the door 72 adjusted to the opened position to provide access to the access opening 58. The valve 76 may be in the closed position and may seal the compartments 20 from the exterior of the housing 68.

The door 72 may be biased to a closed position to seal the access opening 58 and a user may apply a force to the door 72 to adjust the door 72 against the bias and to the opened position. In some examples, the door 72 may be adjusted to the opened position in response to a user engaging the door 72 with the specimen 16. In some configurations, the door 72 may be automated and opened and/or closed in response to a control signal received from the reader device 14 and/or the specimen detector 24 or cartridge detector 30 detecting that the specimen 16 is proximate the cartridge 12 and/or positioned in the cartridge 12. In some configurations, the door 72 may not be biased in any position or direction.

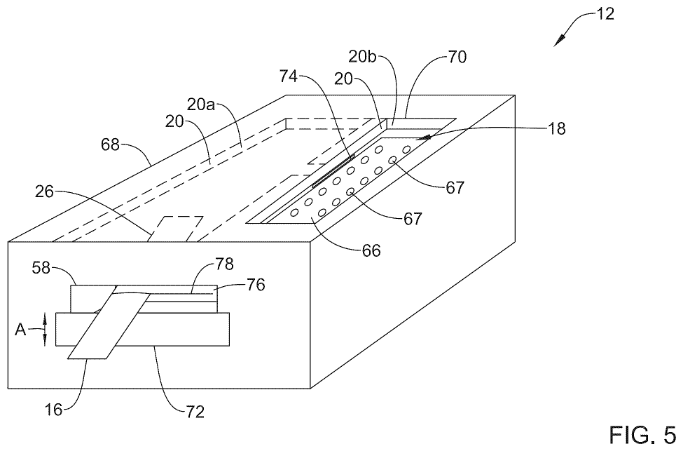

FIG. 5 schematically depicts a perspective view of the configuration of the cartridge 12 depicted in FIG. 4, with the door 72 in the opened position and the specimen 16 inserted into the access opening 58, through the valve 76 and the slit 78, and into the first compartment 20a (e.g., with the sample portion 26 of the specimen 16 at least partially inserted in the first compartment 20a). As depicted in FIG. 5, the valve 76 may seal around the specimen 16 when the specimen 16 is inserted through the slit 78 and/or as the specimen 16 is advanced through the valve 76, but other configurations are contemplated.

The specimen 16 may be any suitable type of specimen configured to extend through the access opening 58 and into the first compartment 20a. As depicted in FIG. 5, the specimen 16 may be a test strip, but other suitable specimens 16 may be utilized including, but not limited to, a swab or a swab on a stick (e.g., Q-tip) and/or other suitable specimens.

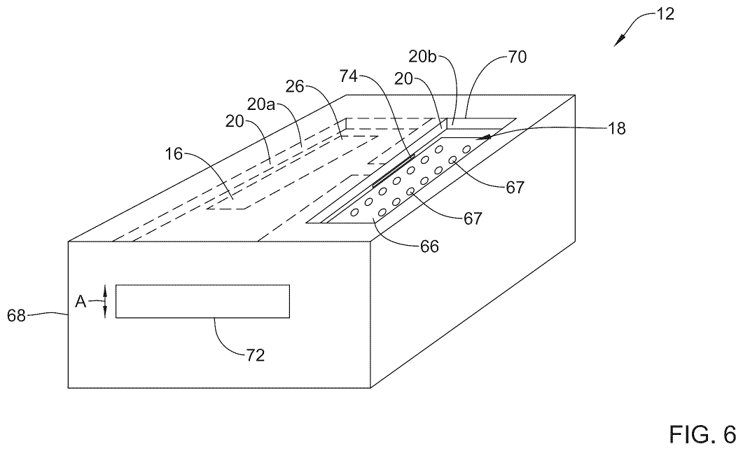

FIG. 6 schematically depicts a perspective view of the configuration of the cartridge 12 depicted in FIG. 4, with the door 72 in the closed position and the specimen 16 fully inserted in the first compartment 20a. Once at least the sample portion 26 of the specimen 16 is inserted (e.g., fully inserted) into the first compartment 20a, fluid may emanate from a sample on the sample portion 26 through the fluid passage 74 and to or over the reactant array 18 of the CSA 66 such that reactants 67 of the reactant array 18 may react to the fluid if an analyte of interest is detected. In some configurations, the fluid within the compartment 20 may be agitated to encourage the fluid to pass from the sample on the sample portion 26 to the reactant array 18 by moving the cartridge 12 and/or the specimen 16 (e.g., the sample portion 26) around, pumping the fluid in the cartridge 12 with a pump (not shown), and/or by agitating the fluid in one or more other suitable manners.

In some examples, the door 72 may be part of a lock of the cartridge 12 (e.g., the lock 22 and/or other suitable lock). For example, once the door 72 moves from an opened position to the closed position, the door 72 may be permanently positioned in the closed position to prevent re-use of the cartridge 12 with a further specimen 16.

In some examples, the door 72 may include a blade surface configured to engage the specimen 16 and sever the specimen 16 into two or more portions (e.g., a non-sample portion and the sample portion 26). For example, a top surface of the door 72 may be a blade surface configured to engage the specimen 16 when it is extending out of the access opening 58 as the door 72 is adjusted from the opened position to the closed position such that the specimen 16 is severed into two portions.

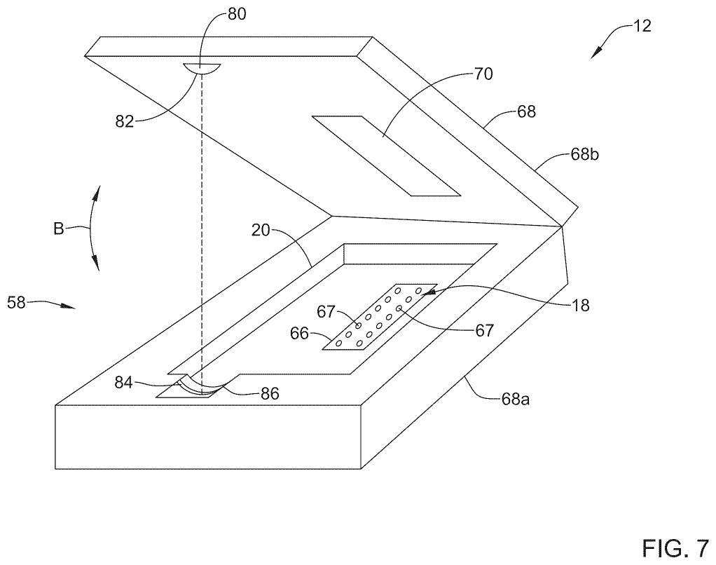

FIGS. 7-9 schematically depict an illustrative configuration of the cartridge 12 including the housing 68 having a first component 68a (e.g., a base) and a second component 68b (a lid or cover), the access opening 58, and a transparent portion 70 (e.g., a window formed from glass, polymer, and/or other suitable transparent material) through which the reader device 14 may view, collect light, and/or image the reactant array 18 positioned within the compartment 20. The first component 68a of the housing 68 may entirely or at least partially define the compartment 20 in which the CSA 66 with the reactant array 18 is located and in which the specimen 16 may be positioned when inserted in the housing 68. The second component 68b of the housing 68 may include the transparent portion 70 and a first connector 80 for coupling with a second connector 84 on the first component 68a of the housing 68. Although not required, the first connector 80 may include a blade surface 82, which may be sharpened or may be blunt, configured to engage and sever a specimen 16 extending out of the cartridge 12.

The first component 68a and the second component 68b of the housing 68 may be adjustably configured to adjust in the directions of arrows B or in other suitable directions between an opened position at which the access opening 58 may be accessible and a closed position at which the access opening 58 is not accessible or is blocked. In some configurations, the first component 68a and the second component 68b may be configured to fluidly seal (e.g., hermetically seal) the access opening 58 and/or the compartment 20 when in the closed position. Although not depicted, one or both of the first component 68a and the second component 68b of the housing 68 may include one or more seals configured to engage the other component of the housing 68 to fluidly seal the access opening 58 and/or the compartment 20.

The housing 68 may include or define one or more compartments 20. As depicted in FIGS. 7-9, the housing 68 may have a single compartment 20 for receiving the sample portion 26 of the specimen 16 and for containing the reactant array 18 (e.g., the reactant array 18 of the CSA 66 or other suitable reactant array 18). Alternatively, the housing 68 may have a plurality of compartments 20 for receiving the sample portion 26 of the specimen 16, containing the reactant array 18, and/or for receiving or containing one or more other components of the cartridge 12.

The first component 68a may define a guide 86 configured to receive a portion of the specimen 16. In some examples, when the specimen 16 is received within the guide 86, the specimen 16 may be aligned at a desired position within the compartment 20 relative to the reactant array 18. In some examples, when the specimen 16 is received within the guide 86, the specimen 16 may be aligned with the first connector 80 and the second connector 84 such that the specimen 16 may be severed at a desired location when the first component 68a and the second component 68b are adjusted to the closed position, the blade surface 82 engages specimen 16, and the first connector 80 and the second connector 84 are coupled to one another.

The first connector 80 and the second connector 84 may be part of a lock of the cartridge 12 (e.g., the lock 22 and/or other suitable lock). For example, once the first component 68a and the second component 68b are adjusted to a closed position, a latch or other feature of the first connector 80 may engage a feature of the second connector 84 to permanently maintain the first component 68a and the second component 68b of the housing 68 in the closed position to prevent re-use of the cartridge 12 with a further specimen 16.

FIG. 7 schematically depicts a perspective view of the cartridge 12 with the first component 68a and the second component 68b of the housing 68 adjusted to the opened position to provide access to the access opening 58 (e.g., for placing the specimen in the compartment 20, removing spent materials form the compartment 20, cleaning the compartment 20, etc.) Although not required, the first component 68a and the second component 68b may be coupled to one another via hinge and may pivot with respect to one another.

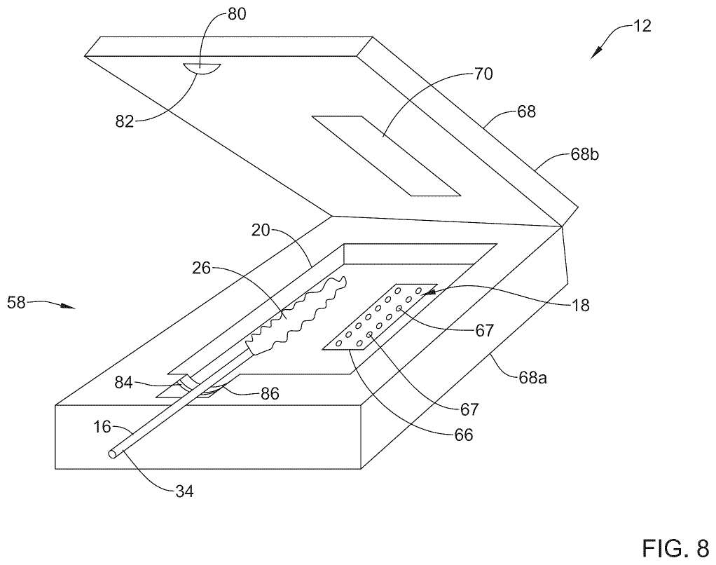

FIG. 8 schematically depicts a perspective view of the configuration of the cartridge 12 depicted in FIG. 7, with the first component 68a and the second component 68b in the opened position and the specimen 16 inserted into the access opening 58 such that the sample portion 26 of the specimen 16 is inserted in the compartment 20 at a desired location proximate (e.g., in fluid communication with) the reactant array 18. As depicted in FIG. 8, a non-sample portion 34 of the specimen 16 may be positioned within the guide 86 to facilitate positioning the sample portion 26 in the compartment 20 and aligning the non-sample portion 34 with the blade surface 82.

The specimen 16 may be any suitable type of specimen configured to extend through the access opening 58 and into the compartment 20. As depicted in FIG. 8, the specimen 16 may be a swab on a stick (e.g., a Q-tip) and/or one or more other suitable specimens.

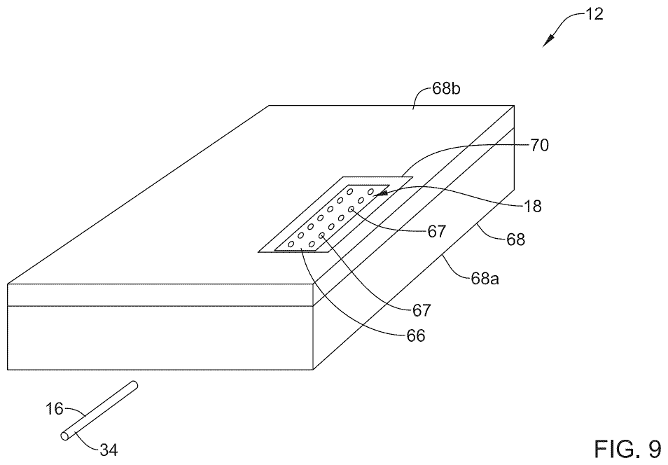

FIG. 9 schematically depicts a perspective view of the configuration of the cartridge 12 depicted in FIG. 7, with the first component 68a and the second component 68b in the closed position, with the sample portion 26 of the specimen 16 in the compartment 20 (not shown) and a severed non-sample portion 34 exterior of and separated from the cartridge 12. Once the sample portion 26 of the specimen 16 is inserted and enclosed into the compartment 20, fluid may emanate from a sample on the sample portion 26 to or over the reactant array 18 of the CSA 66 such that the reactants 67 of the reactant array 18 may react to the fluid if an analyte of interest is detected. In some configurations, the fluid within the compartment 20 may be agitated to encourage the fluid to pass from the sample on the sample portion 26 to the reactant array 18 by moving the cartridge 12 around, pumping the fluid in the cartridge 12 with a pump (not shown), and/or by agitating the fluid in one or more other suitable manners.

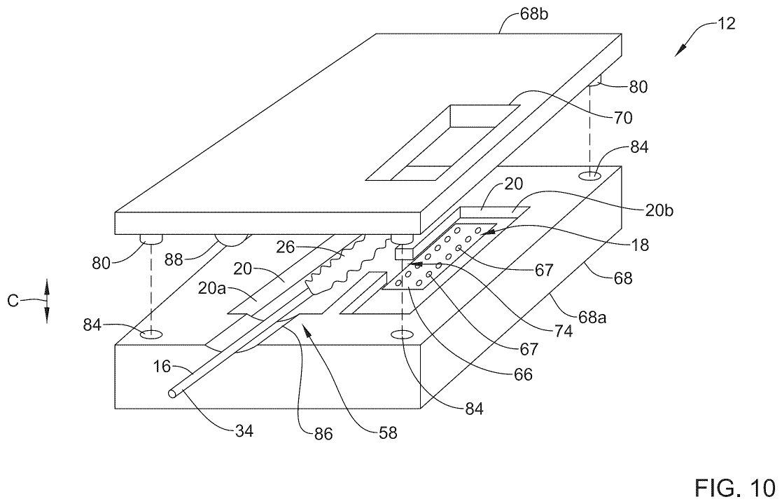

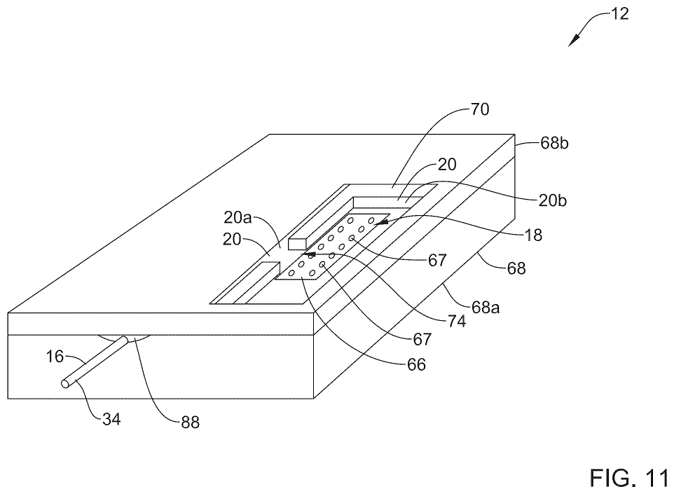

FIGS. 10 and 11 schematically depict an illustrative configuration of the cartridge 12 including the housing 68 having a first component 68a (e.g., a base) and a second component 68b (a lid or cover), the access opening 58, and a transparent portion 70 (e.g., a window formed from glass, polymer, and/or other suitable transparent material) through which the reader device 14 may view, collect light, and/or image the reactant array 18 positioned within the compartment(s) 20. The first component 68a of the housing 68 may entirely or at least partially define the compartment(s) 20 in which the CSA 66 with the reactant array 18 may be located and in which the specimen 16 may be positioned when inserted in the housing 68. The second component 68b of the housing 68 may include the transparent portion 70 and first connectors 80 (e.g., pegs or legs) for coupling with second connector 84 (e.g., holes or openings) on the first component 68a of the housing 68.

First component 68a and the second component 68b of the housing 68 may be adjustably configured to adjust in the directions of arrow C or in other suitable directions between an opened position at which the access opening 58 may be accessible and a closed position at which the access opening 58 is not accessible or is blocked. In some configurations, the first component 68a and the second component 68b may be configured to fluidly seal (e.g., hermetically seal) the access opening 58 and/or the compartment(s) 20 when in the closed position. Although not depicted, one or both of the first component 68a and the second component 68b of the housing 68 may include one or more seals configured to engage the other component of the housing 68 to fluidly seal the access opening 58 and/or the compartment(s) 20.

The housing 68 may include or define one or more compartments 20. As depicted in FIGS. 9 and 10, the first component 68a of the housing 68 may have a first compartment 20a for receiving the sample portion 26 of the specimen 16 and a second compartment 20b for containing the reactant array 18 (e.g., a reactant array 18 of the CSA 66 or other suitable reactant array 18). When two or more compartments 20 are utilized in the housing 68, the compartments 20 may be fluidly connected by one or more fluid passages 74 such that fluid may pass from one compartment 20 (e.g., the first compartment 20a) to another (e.g., the second compartment 20b).

The first component 68a of the housing 68 may define a guide 86 configured to receive a portion of the specimen 16. In some examples, when the specimen 16 is received within the guide 86, the specimen 16 may be aligned at a desired position within the first compartment 20a relative to the reactant array 18 in the second compartment 20b and the fluid passage 74.

The second component 68b of the housing 68 may include a resilient seal 88 aligned with the guide 86. When the first component 68a and the second component 68b of the housing 68 are in the closed position and the specimen 16 (e.g., the non-sample portion 34 of the specimen 16) is in the guide 86, the resilient seal 88 may extend around and adjust to a shape of the specimen 16 to create a fluid-tight seal around the specimen 16 when the housing 68 is in the closed configuration.

In some examples, the first connectors 80 and the second connectors 84 may be part of a lock of the cartridge 12 (e.g., the lock 22 and/or other suitable lock). For example, once the first components 68a and the second component 68b of the housing 68 are adjusted to a closed position, the first connectors 80 may engage a feature of the second connector 84 (e.g., via a friction fit, a snap, a latch, etc.) to permanently maintain the first component 68a and the second component 68b of the housing 68 in the closed position and prevent re-use of the cartridge 12 with a further specimen 16.

FIG. 10 schematically depicts a perspective view of the cartridge 12 with the first component 68a and the second component 68b adjusted to the opened position to provide access to the access opening 58 and with the specimen 16 inserted into the access opening 58 such that the sample portion 26 of the specimen 16 is in the first compartment 20a at a desired location proximate (e.g., in fluid communication with) the reactant array 18 and/or the fluid passage 74. As depicted in FIG. 10, a non-sample portion 34 of the specimen 16 may be positioned within the guide 86 to facilitate positioning the sample portion 26 in the first compartment 20a and aligning the non-sample portion 34 with the resilient seal 88.

The specimen 16 may be any suitable type of specimen configured to extend through the access opening 58 and into the compartment 20. As depicted in FIG. 10, the specimen 16 may be a swab on a stick (e.g., a Q-tip) and/or one or more other suitable specimens.

FIG. 11 schematically depicts a perspective view of the configuration of the cartridge 12 depicted in FIG. 10, with the first component 68a and the second component 68b in the closed position, with the sample portion 26 of the specimen 16 in the first compartment 20a (not shown), and with the non-sample portion 34 extending from the guide 86 and exterior of the cartridge 12. The resilient seal 88 may extend into or about the guide 86, engage the specimen 16, and fluidly seal and maintain a position of the specimen 16 with cartridge 12. Once the sample portion 26 of the specimen 16 is inserted and enclosed into the first compartment 20a, fluid may emanate from a sample on the sample portion 26, through the fluid passage 74, and to or over the reactant array 18 of the CSA 66 in the second compartment 20b such that the reactants 67 of the reactant array 18 may react to the fluid if an analyte of interest is detected. In some configurations, the fluid within the first compartment 20a, the fluid passage 74, and/or the second compartment 20b may be agitated to encourage the fluid to pass from the sample on the sample portion 26 to the reactant array 18 by moving the cartridge 12 around, pumping the fluid in the cartridge 12 with a pump (not shown), and/or by agitating the fluid in one or more other suitable manners.

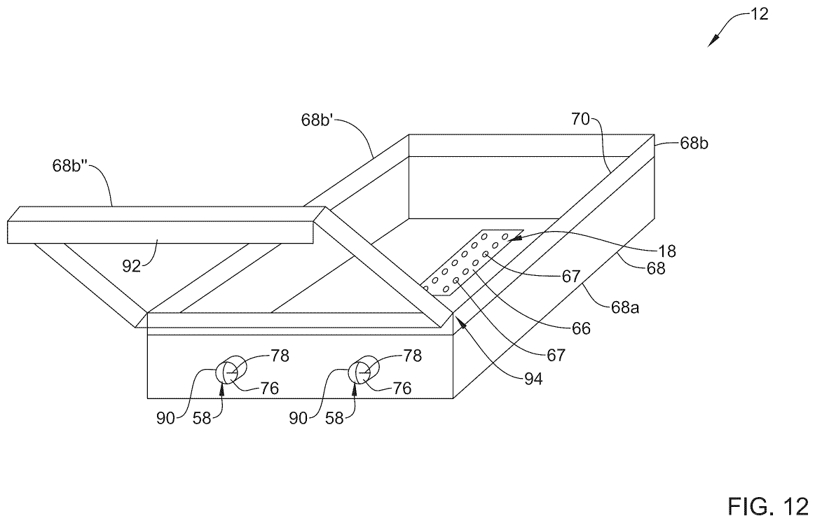

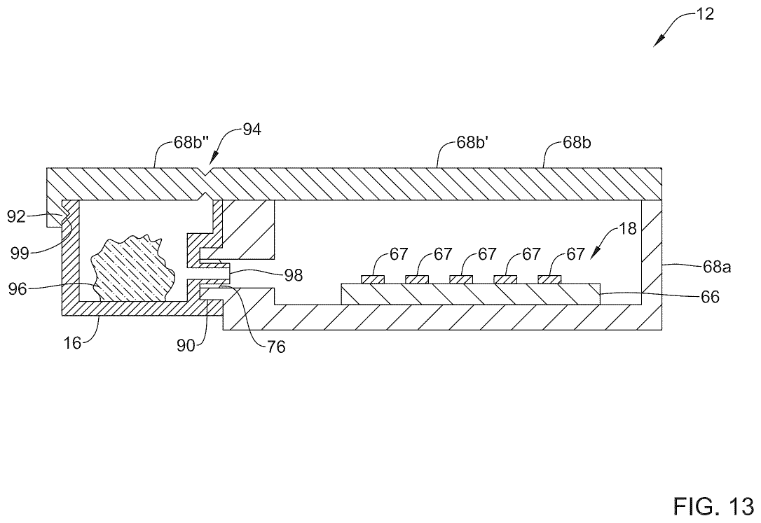

FIGS. 12 and 13 schematically depict an illustrative configuration of the cartridge 12 including the housing 68 having a first component 68a (e.g., a base) and a second component 68b (a lid or cover), access openings 58, and a transparent portion 70 (e.g., an entirety or at least a portion of the second component 68b of the housing 68 formed from glass, polymer, and/or other suitable transparent material) through which the reader device 14 may view, project light onto, collect light from, and/or image the reactant array 18 positioned within the compartment(s) 20. The first component 68a of the housing 68 may entirely or at least partially define the compartment(s) 20 in which the CSA 66 with the reactant array 18 is located and in which fluid from the specimen 16 may travel when the specimen is in fluid communication with the compartment(s) 20.

The second component 68b of the housing 68 may be releasably or permanently coupled with the first component 68a of the housing 68. When the first component 68a and the second component 68b are coupled with one another, a seal between the components 68a, 68b may be a hermetic seal.

The first component 68a of the housing 68 may include one or more cartridge ports 90 (e.g., two cartridge ports 90, as depicted in FIG. 12, or other suitable number of cartridge ports 90) configured to engage the specimen 16. In operation, the cartridge ports 90 may be configured to couple with tubing and/or a specimen 16, where the specimen 16 may be container containing a sample and the container may be configured to couple with the cartridge ports 90

The second component 68b includes a first portion 68b′ that may cover or act as a lid for the first component 68a of the housing 68 and a second portion 68b″ that is configured to engage and, optionally, act as a cover for the specimen 16 coupled with the first component 68a of the housing 68. The second portion 68b″ of the second component 68b may extend from the first portion 68b′ via living hinge 94 or other suitable hinge and pivot relative to the first portion 68b′. In some configurations, the second portion 68b″ may include a latch 92 or other feature configured to engage the specimen 16 when the specimen 16 is coupled with the cartridge ports 90.

One or more valves 76 may be positioned at or in the access openings 58 extending through the cartridge ports 90. In some examples, as depicted in FIG. 12, a single valve 76 (e.g., a check valve and/or other suitable type of valve) may be located in the access opening 58 of each cartridge port 90, where the single valve 76 may include a slit 78 or other opening biased closed and that may be opened in response to engagement with the specimen 16. When the specimen ports of the specimen 16 extend through the valves 76, the valves 76 may be configured to seal around the specimen ports. Alternatively or additionally, the cartridge ports 90 may be covered with peel-way or puncturable seals.

The first portion 68b′ of the second component 68b of the housing 68 and the first component 68a may be fixedly sealed with respect to one another, but this is not required and the first component 68a and the second component 68b of the housing 68 may be adjustable with respect to one another to provide access to the compartment(s) 20. When the first component 68a and the second component 68b are sealed relative to one another, the seal may be a fluid-tight (e.g., hermetic) seal.

FIG. 12 schematically depicts a perspective view of the cartridge 12 with the second portion 68b″ of the second component 68b adjusted to an opened position to receive the specimen 16 and provide access to the access openings 58. In the opened position, the second portion 68b″ may be rotated upward to facilitate the specimen 16 engaging the cartridge ports 90. Alternatively, the cartridge 12 may include the specimen 16 (e.g., in a container or carrier configuration) and the second portion 62b″ arranged in the opened position may facilitate receiving the sample 96 in the specimen 16.

FIG. 13 schematically depicts a cross-section view of the configuration of the cartridge 12 depicted in FIG. 12, with the specimen 16 (e.g., in a container configuration) engaging the cartridge ports 90 of the first component 68a of the housing 68. Although the specimen 16 is depicted as being connectable to the housing 68 and a separate component therefrom, the specimen 16 may be part of and/or a permanent structure of the housing 68, as discussed.

The specimen 16 may include one or more specimen ports 98 configured to engage and/or extend through the cartridge ports 90, as depicted in FIG. 13. In some examples, the specimen ports 98 may be configured to extend through the valves 76 in the access openings 58 and the valves 76 may seal around the specimen ports 98.

Before, during, or after the specimen 16 is coupled with the cartridge ports 90, the second portion 68b″ of the second component 68b may be adjusted and coupled with the specimen 16. In some examples, the second portion 68b″ may couple with the specimen 16 via a snap connection (e.g., a permanent connection or reversible connection) between the latch 92 and a notch 99 in the specimen 16. Although not required, the second portion 68b″ coupled with the specimen 16 may act as a cover to the specimen 16 such that fluid from a sample 96 in the specimen 16 may only leave the specimen 16 via the specimen ports 98.

Once the specimen 16 is securely coupled with the cartridge 12 and in fluid communication with the compartment 20, fluid may emanate from the sample 96 in the specimen 16, through the specimen port(s) 98, through the cartridge port 90, and to or over the reactant array 18 of the CSA 66 in the second compartment 20b such that the reactants 67 of the reactant array 18 may react to the fluid if an analyte of interest is detected. In some configurations, the fluid within the compartment 20 and/or the specimen 16 may be agitated to encourage the fluid to pass from the sample 96 to the reactant array 18 by moving the cartridge 12 around, pumping the fluid in the cartridge 12 with a pump (not shown), and/or by agitating the fluid in one or more other suitable manners.

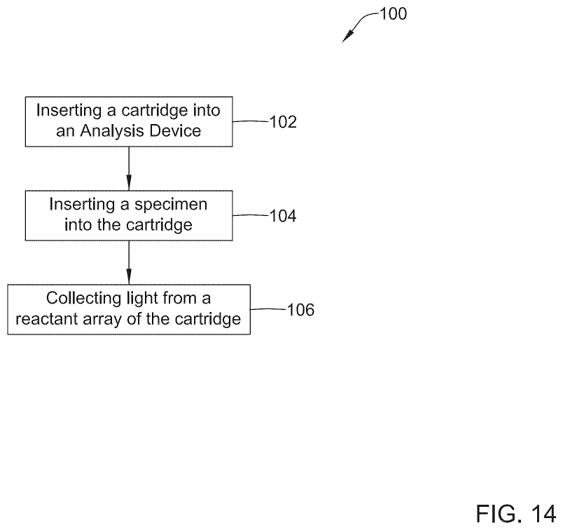

FIG. 14 schematically depicts an illustrative method 100 that may facilitate analyzing a reactant array (e.g., a reactant array of a CSA or other suitable reactant array) exposed to a fluid from a sample (e.g., as part of a fluid analysis test on one or more fluids of interest). The method 100 may include inserting 102 a cartridge into a device (e.g., a reader device) configured to analyze reactant arrays. In some configurations, the cartridge may include a CSA including a reactant array configured to be exposed to a fluid and an opening configured to receive and/or engage a specimen with a sample thereon. The device may be configured to collect light from the reactant array through the cartridge and analyze the collected light instantaneously and/or changes in the collected light over time.

Further, the method 100 may include inserting 104 a specimen into the cartridge. In some cases, the specimen may be positioned or may be in fluid communication with a compartment of the cartridge, where the compartment may be in fluid communication with the reactant array of the CSA. In some examples, the specimen may include a sample portion having a sample thereon from which fluid may exude or be emitted to the reactant array. When included, the sample portion of the specimen may be positioned in the compartment of the cartridge as the specimen is inserted into the cartridge and/or at one or more other suitable times. The sample may be collected from an area of interest by applying the specimen to the area of interest (e.g., a wound, pollen from a flower, an infection, an exhalation from a subject, a sweat gland, etc.)

In some configurations, as the specimen is inserted into and secured within the cartridge, the specimen may be locked in the cartridge. As such, the cartridge may be prevented from being used a second time with one or more other specimens. In some examples, the reader device that receives the cartridge may be able to detect if the cartridge and/or the specimen has been previously used, as discussed herein or otherwise, and if the cartridge and/or specimen are determined to have been used, the reader device may reject performing a fluid analysis test using the specimen and/or cartridge.

Although not required, light from one or more light sources may be applied to the reactants of the reactant array. The light may be applied directly to the reactants of the reactant array and/or through a transparent portion of the substrate. Application of light to the reactants of the reactant array may facilitate collection of light from the reactant array as the reactants are exposed to fluid.

The method 100 may include collecting 106 light from the reactant array of the CSA. The light from the reactant array may be collected with the reader device using a light or image sensor. Although not required, the light may be collected from the reactant array while light is being applied to the reactant array.

In some configurations, the reader device may be configured to analyze levels of wavelengths of light collected from the reactant array. The levels of the wavelengths of light collected from the reactant array may be measured in any suitable manner including, but not limited to, by counting photons at one or more wavelengths of light collected, measuring an amount of light collected at one or more wavelengths of light collected, a change in photon count over time for one or more wavelengths of light collected, a change in pixel value (e.g., a change in pixel grayscale value) of an image sensor over time, and/or levels of wavelengths of light collected may be measured in one or more other suitable manners.

The measurements of the levels of the wavelengths of light collected from the reactant array may be utilized to identify an analyte (e.g., component) of the fluid to which the reactant array may be exposed. In some examples, when the levels of the wavelengths of light collected over time match or closely resemble a set of expected wavelength levels of the reactant(s) exposed to a known analyte (e.g., a fluid or component of fluid), the known analyte may be identified as being present in the fluid tested in the fluid analysis test. Example techniques for measuring levels of wavelength of light collected from reactant arrays and for comparing measurements to known measurements associated with fluids are discussed in U.S. Patent Application No. PCT/US2023/083024 (Attorney docket no. 1519.1004111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for analyzing reactant arrays, the system comprising:
a cartridge, the cartridge includes a reactant array, a compartment for receiving a specimen that is in fluid communication with the reactant array, and an access opening configured to receive a sample portion of the specimen; and
a device for analyzing the reactant array when the cartridge is received in the device.

2. The system of claim 1, wherein the cartridge comprises:
a housing at least partially enclosing the reactant array, defining the compartment for receiving the specimen, and the access opening through which the specimen is inserted into the compartment.

3. The system of claim 2, wherein the housing comprises a first component and a second component adjustable relative to one another between an opened position to define the access opening and a closed position to block the access opening.

4. The system of claim 1, wherein the housing is formed from a single housing component.

5. The system of claim 1, wherein the cartridge includes a lock having a locked position to prevent removal of the specimen from the cartridge.

6. The system of claim 5, wherein the lock is automatically adjusted from an unlocked position to the locked position in response to insertion of the specimen in the cartridge.

7. The system of any one of claims 1-6, wherein the device is configured to automatically detect insertion of the cartridge in the device.

8. The system of claim 1, wherein the device is configured to automatically detect insertion of the specimen in the cartridge.

9. The system of claim 1, wherein the device is configured to collect light from the reactant array in response to the cartridge being positioned in the device.

10. The system of claim 1, further comprising:
the specimen, wherein the specimen includes the sample portion for positioning in the compartment and a non-sample portion extending out of the compartment.

11. The system of claim 10, wherein the cartridge is configured to sever a portion of the non-sample portion from the sample portion.

12. A cartridge for use with a device for analyzing reactant arrays, the cartridge comprising:
a reactant array;
a housing at least partially enclosing the reactant array, the housing defining a compartment for receiving a specimen; and
an access opening extending from exterior of the housing into the compartment for receiving the specimen.

13. The cartridge of claim 12, wherein the housing comprises a first component and a second adjustable relative to one another between an open position to define the access opening and a closed position to block the access opening.

14. The cartridge of claim 12, wherein the access opening is a through-hole extending from an exterior surface of the housing to the compartment for receiving the specimen.

15. The cartridge of claim 14, wherein the through-hole is configured to receive the specimen and the specimen comprises a swab.

16. The cartridge of claim 12, further comprising:
a lock configured to prevent removal of the specimen from the housing.

17. The cartridge of claim 16, wherein the lock is configured to adjust from an unlocked position to a locked position in response to receiving the specimen in the compartment.

18. The cartridge of claim 12, further comprising:
a blade configured to sever the specimen received in the compartment.

19. The cartridge of claim 12, wherein the cartridge is configured to fluidly isolate the compartment and the reactant array from fluid exterior of the housing.

20. A method of analyzing reactant arrays, the method comprising:
inserting a cartridge into a device, where the cartridge includes a compartment and a reactant array and the device is configured to analyze the reactant array by collecting light through the cartridge;
inserting a specimen into the cartridge, where the specimen includes a sample portion, the compartment is in fluid communication with the reactant array, and the sample portion is positioned in the compartment of the cartridge; and
collecting, with the device, light from the reactant array after the cartridge is inserted into the device.

21. The method of claim 20, further comprising:
locking the specimen in the cartridge in response to receiving the specimen in the cartridge.
